(19) United States
(12) Patent Application Publication
(10) Pub. No.: US 2004/0102710 A1
(43) Pub. Date: May 27, 2004

(54) Method of wave form segmentation and characterization of the segmented interval thereof

(76) Inventor: Jungkuk Kim, Sung-Mam City (KR)

Correspondence Address: Volentine Francos, Suite 150, 12200 Sunrise Valley Drive, Reston, VA, 20191, US

(21) Appl. No.: 10/451,700

(22) PCT Filed: Jul. 8, 2002
(86) PCT No.: PCT/KR02/01287

(30) Foreign Application Priority Data
Nov. 13, 2001 (KR) 2001/70371

(51) Int. Cl. A61B 5/04

(52) U.S. Cl. 600/509

(57) ABSTRACT

The present invention discloses a method of partitioning a waveform for characterization with a slope-inversion point and a slope-transition point by utilizing a slope-tracing waveform, which can be utilized for the application to the physiological signal of a living body.

### FIELD OF THE INVENTION

The present invention relates to a method of partitioning a signal waveform and characterizing the section partitioned thereof, and more particularly, to a method of dividing a signal waveform into several sections, which is appropriate for recognizing the signal recognition through a mathematical integration of the waveform between a slope-inversion point and a slope-transition point.

### BACKGROUND ART

The present invention can find its application in the area of the recognition of a wide range of signal waveforms including physiological signal of a living body such as ECG (electrocardiography), EEG (electroencephalography), EMG (electromyography), electrogram, endocardiogram, and pulsation waveform.

Traditionally, various approaches have been tried to partition a continuous time-varying signal waveform into sections. More often, a section of a signal waveform is defined as an interval between two curvature-transitioning points where the curvature of the waveform changes its polarity.

The prior art, however, has a shortcoming in a sense that the density of partitioned sections tends to be excessively high if the signal waveform varies quite rapidly with respect to time.

Furthermore, the prior art has a limit because a couple of successive waveforms, for instance, in the case of physiological waveforms of a living body, are erroneously interpreted as a single continuous waveform.

### DETALED DESCRIPTION OF THE INVENTON

Accordingly, it is an object of the present invention to provide a method of efficiently partitioning a signal waveform into sections and characterizing the sections partitioned thereof.

It is further an abject of the preset invention to provide a method of partitioning a signal waveform, which is appropriate to be applied for physiological signals of a living body, through utilizing a slope-inversion point and a slope-transition point.

In order to accomplish the above-mentioned objects, the present invention provides a method of partitioning a signal waveform comprising steps of (a) updating the functional value of an (n+1)-th sample with an amplitude of an (n+1)-th sample if the functional value of an n-th sample of a tracing waveform is less than the amplitude of an (n+1)-th sample of a signal waveform; (b) comparing the functional value of an n-th sample of said tracing waveform with the functional value of an (n-1)-th sample of said tracing waveform if the functional value of an n-th sample of said tracing waveform is either greater than or equal to the amplitude of an (n+1)-th sample of said signal waveform; (c) maintaining the functional value of an (n+1)-th sample of said tracing waveform with a functional value of an n-th sample in case when the functional value of an n-th sample of said tracing waveform is the same as the functional values of (n−1)-th, (n−2)-th, (n−1)-th samples wherein 1 is less than k, a predetermined number, and updating an (n+1)-th sample of said tracing waveform by subtracting an amount with an average slope between the amplitude of an (n-k)-th sample and the amplitude of an n-th sample in case when the functional value of an n-th sample of said tracing waveform is the same as the functional values of (n−1)-th, (n−2)-th, . . . , (n−1)-th samples wherein 1 is equal to k at the step of (b); (d) updating the value of an (n+1)-th sample by subtracting an amount with the same slope from (referred as a first slope) the value of an n-th sample if the functional value of said n-th sample of said tracing waveform is different from those of (n−1)-th, (n−2)-th, (n−j)-th samples wherein j is less than L, a predetermined value), and updating the value of an (n+1)-th sample by subtracting an amount with an average slope between an (n−L)-th sample of said signal waveform and an n-th sample of the signal waveform (referred to as “a second slope”) if said second slope is steeper than said first slope by a predetermined amount rate (X %) and if the functional value of said n-th sample of said tracing waveform is different form these of (n−1)-th, (n−2)-th, . . . , (n−j)-th samples wherein j is equal to L), and updating the value of an (n+1)-th sample by subtracting an amount with a new slope that is produced by multiplying a predetermined rate (X %) to said first slope if the value said second slope is smaller than that of the multiplication of said first slope by said predetermined rate (X %); and (e) recognizing an (n+1)-th sample as a slope-transition point and considering the interval between said slope-inversion point and said slope-transition point as a single interval if the functional value of an (n+1)-th sample of said tracing waveform is less than or equal to the amplitude of an (n+1)-th sample of said signal waveform, thereby said two waveforms crossing.

### BRIEF DESCRIPTION OF THE DRAWINGS

Further feature of the present invention will become apparent from a description of a method of partitioning a signal waveform into sections with a slope-inversion point and a slope-transition point through a tracing waveform taken in conjunction with the accompanying drawings of an embodiment of the invention, which, however, should not be taken to be limitative to the invention, but are for explanation and understanding only.

In the drawings:

FIG. 1 is a schematic diagram illustrating a preferred embodiment of a lower slope-tracing waveform with a signal waveform for the partition of the waveform into sections in accordance with the present invention.

FIG. 2 is a schematic diagram illustrating how a lower slope-tracing waveform chases a signal waveform during the ascending stage where the amplitude of the signal waveform increases in accordance with the present invention.

FIG. 3 is a schematic diagram illustrating the behavior of a lower slope-tracing waveform during the descending stage posterior to a slope-inversion point in accordance with the present invention.

FIGS. 4A though 4C are schematic diagrams illustrating the effect when the number of samples is varied for keeping the lower slope-tracing waveform constant after a slope-inversion point has been detected, in accordance with the present invention.

FIG. 5 is a schematic diagram illustrating a preferred embodiment wherein a slope-transition point is determined with a lower slope-tracing waveform and thereby the signal waveform in partitioned.

FIG. 6 is a schematic diagram illustrating an upper slope-tracing waveform with a signal waveform for partitioning the signal waveform into sections in accordance with the present invention.

FIG. 7 is a schematic diagram illustrating the behavior of an upper slope-tracing waveform during an ascending stage where a signal waveform increases to a maximum in accordance with the present invention.

FIG. 8 is a schematic diagram illustrating the behavior of an upper slope-tracing waveform at descending stage posterior to a slop-inversion point in accordance with the present invention.

FIGS. 9A through 9C are schematic diagrams illustrating the effect when the number of samples is changed for keeping the upper slope-tracing waveform constant after a slope-inversion point is detected, in accordance with the present invention.

FIG. 10 is a schematic diagram illustrating a preferred embodiment wherein a slope-transition point is determined with an upper slope-tracing waveform and thereby the signal waveform is partitioned.

FIG. 11 is a schematic diagram illustrating a signal waveform with partitioned sections using a lower slope-tracing waveform.

FIG. 12 is a schematic diagram illustrating a waveform with partitioned sections using an upper slope-tracing waveform.

### DETAILED DESCRIPTION OF THE PREFERRED EMBODIMENT OF THE INVENTION

Features of the present invention will be explained in detail with reference the accompanying drawings.

<Description of Terminology>

Slope-inversion point: a point of a sampled signal waveform where the waveform switches the polarity of its slope or the differential derivative either from the negative to the positive or from the positive to the negative.

Slope-transition point: a point where the slope of a signal waveform changes very rapidly. Here, the degree of the rapidness in the change of slope can be understood in a sense that the rate of slope-change at a certain point is larger than a predefined value (X %). As a preferred embodiment, X can be chosen as 50%.

Slope-tracing waveform: a waveform that is chasing a signal waveform and is employed for efficiently determining the slope-transition point and the slope-inversion point. Two types of slope-tracing waveform are disclosed as a preferred embodiment: one is a lower slope-tracing waveform which traces a signal waveform upward from the beneath, and the other is an upper slope-tracing waveform which traces a signal waveform downward from the top.

Preferably, both the upper and lower slope-tracing waveforms can be simultaneously employed for partitioning the signal waveform. Depending upon a situation, either the upper slope-tracing waveform or the lower slope-tracing waveform can be chosen.

<Determination of a Slope-Inversion Point and a Slope-Transition Point with a Lower Slope-Tracing Waveform>

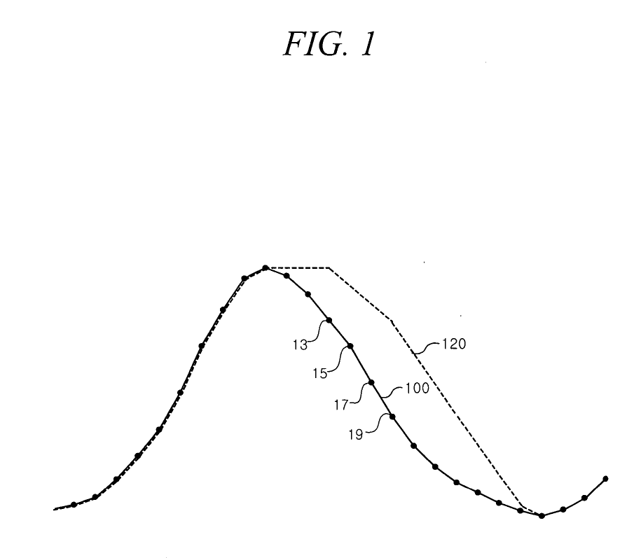

FIG. 1 is a schematic diagram illustrating a lower slope-tracing waveform with a signal waveform for partitioning the signal waveform into sections in accordance with the present invention.

Referring to FIG. 1, the solid line 100 represents a signal waveform to be partitioned while the dotted line 120 denotes a curve of a lower slope-tracing waveform.

The behavior of the lower slope-tracing waveform, as shown in FIG. 1, can be classified as two cases depending upon the relative magnitude of the amplitude between the signal waveform an the slope-tracing waveform.

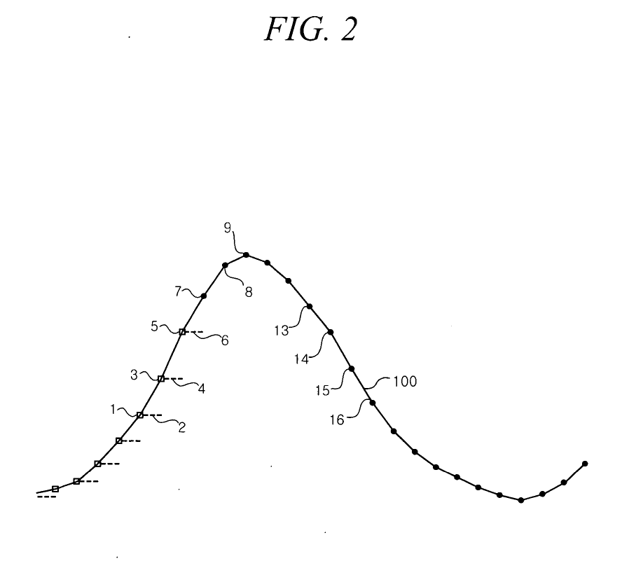

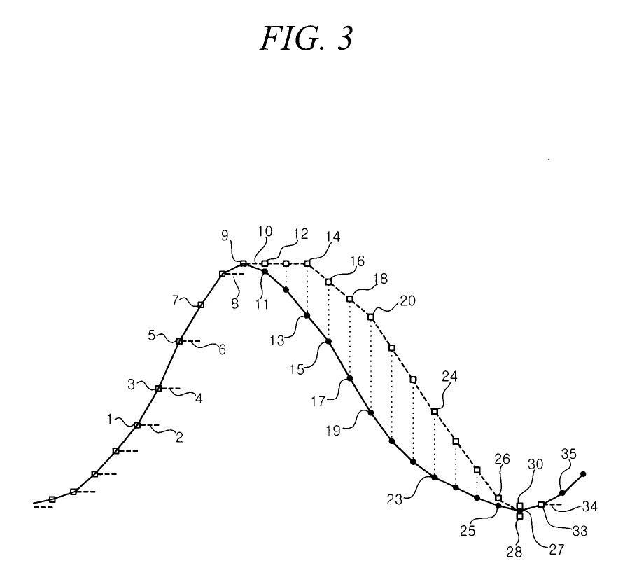

FIG. 2 illustrates a case when the amplitude of the signal waveform is greater than that of the slope-tracing waveform, while FIG. 3 corresponds to a case when the amplitude of the signal waveform is less than that of the slop-tracing waveform.

Additionally, the dots • 13, 15, 17, 19 depicted in FIG. 1 represent the functional values of the samples or the amplitudes of sampling points under consideration.

FIG. 2 is a schematic diagram illustrating the behavior of a lower slope-tracing waveform during the ascending stage where a signal waveform increases in accordance with the present invention.

Namely, FIG. 2 exhibits a case when the amplitude of a signal waveform is greater than that of a lower slope-tracing waveform.

Referring to FIG. 2, the waveform represented by a solid line 100 is a signal waveform which needs to be partitioned, while the sampled dots • 13, 14, 15, 16 represent the functional values of samples or the amplitude at an instant under consideration prior to the application of the lower slope-tracing waveform.

Moreover, the rectangles □ 1, 3, 5 denote the position of the lower slope-tracing after the samples are produced, while the dotted lines 2, 4, 6 denote the height of the lower slope-tracing waveform prior to sampling.

In case when the amplitude of the signal waveform 100 is greater than the height of the lower slope-tracing waveform, the lower slope-tracing waveform is updated with the signal waveform.

Let us choose the seventh sample 5 as a sample under consideration for the explanation purposes. In this case it should be noted that the amplitude of the signal waveform at the sample 5 is higher than the height 4 of the lower slope-tracing waveform.

As a consequence, the height of the lower slope-tracing waveform is updated with the amplitude 5 of the signal waveform, followed by a step of comparing the height 6 of the lower slope-tracing waveform with the amplitude of a next sample 7.

Moreover, the height 4 of the lower slope-tracing waveform prior to the current updating process has been updated with the amplitude 4 of the signal waveform because the amplitude 3 of the signal waveform was higher than the height of the slope-tracing waveform. This updating procedure continues until a slope-inversion point 9 is detected as long as the amplitude of the signal waveform at a sample is greater than the height of the lower slope-tracing waveform.

FIG. 3 is a schematic diagram illustrating the behavior of a lower slope-tracing waveform during the descending stage posterior to the slope-inversion point in accordance with the present invention.

Referring to FIG. 3, we can understand how the lower slope-tracing waveform behaves during the descending stage after the slope-inversion point 9 is detected.

When a slope-inversion point 9 is detected, the amplitude of the signal waveform at slope-inversion point 9 is compared with the height 8 of the lower slope-tracing waveform. In this case, since the amplitude 9 of the signal waveform at slope-inversion point 9 is greater than the height 8 of the lower slope-tracing waveform, the height 10 of the lower slope-tracing waveform is updated with the amplitude 9 of the signal waveform.

Thereafter, since the amplitude of the next succeeding sample 11 is lower than the height 10 of the lower slope-tracing waveform, the lower slope-tracing waveform maintains its height 10 up to next sample 13.

The height 14 of the lower slope-tracing waveform is maintained with the amplitude 10 of the signal waveform at the slope-inversion point from the inversion point 9 to the third sample 13 if the amplitude of the signal waveform at any of the aforementioned three successive samples exceeds the height of the lower slope-tracing waveform, and the previously determined slope-inversion point is disregarded.

In the meanwhile, if the amplitude of the signal waveform at the third sample 13 as well as the two preceding samples, three of which follow the slope-inversion point 9 in a successive manner, does not go over the height 12, 14 of the lower slope-tracing waveform, either the difference in the slope or the amplitude between the slope-inversion point 9 and the third sample 13 is calculated and divided by three in order to get an average slope per sample.

Now, the height of the lower slope-tracing waveform is updated with a new value 16 by subtracting an amount from the old value 14 with the average slope per sample. Preferably, the average slope (or the amplitude) can be regarded as the difference of the height (or the amplitude) between the old lower slope-tracing waveform 14 and the updated lower slope-tracing waveform 16.

Now, for the next succeeding sample 15, the amplitude of the sample 15 is compared with the height 16 of the lower slope-tracing waveform.

Since the height 18 of the lower slope-tracing waveform exceeds the amplitude 15 of the signal waveform, the lower slope-tracing waveform is updated with a new value 18 by subtracting an amount with the average slope per sample.

Additionally, since the height 18 of the lower slope-tracing waveform is still higher than the amplitude 17 of the signal waveform at the next sample, the lower slope-tracing waveform is updated again with a new value 20 by subtracting an amount with the average slope per sample.

In this case, the height of the lower slope-tracing waveform updated is compared again with the amplitude 19 at the subsequent sample, and since the amplitude 19 of the signal waveform is still lower than the height of the lower slope-tracing waveform, the height of the lower slope-tracing waveform is reduced once again by the average slope per sample.

As a preferred embodiment, the average slope per sample can be updated with new number, which is defined as a difference between the maximum and the minimum, partitioned by three among the four successive samples after the slope-inversion point.

In other words, the difference between the third sample 13 and the sixth sample 19 is calculated and divided by three for a new average slope per sample. New average slope per sample is then employed for the calculation of the lower slope-tracing waveform up to the next three samples 23.

This procedure continues until the amplitude of the signal waveform happens to exceed the height of the lower slope-tracing waveform.

In the foregoing explanation, the numbers of samples were chosen to be three for the calculation of a new average slope per sample.

However, it does not have to be three, and the number of grouping samples can be changed in consideration of the computing speed.

More preferably, if a new average slope per sample, which has been calculated for the most recent three samples, is lower than the previous one by X percent, the average slope per sample can be updated as X percent of the previous average slope. In FIG. 3 is shown the case when X is equal to 50.

Referring to FIG. 3, the height 26 of the lower slope-tracing waveform is calculated by subtracting the average slope, which is the difference between the maximum 19 and the minimum 23 partitioned by three, multiplied by three from the height 24 of the lower slope-tracing waveform. In this case, since the average slope per sample was employed for the three successive samples, it should be updated with a new value by finding a difference between finding a difference between a maximum 23 and a minimum 25 and partitioning the difference by three.

In the meanwhile, the updated average slope per sample is a small value because the difference between a maximum 23 and a minimum 25 is not big.

Even if the height of the lower slope-tracing waveform should be updated with a height 30, which is calculated by subtracting a new average slope, for the comparison with the next sample 27, the calculated average slope is neglected because it is smaller than so percent of the previous average slope. Therefore, the height of the lower slope-tracing waveform is now updated with the lower value 28 by using a number of 50 percent of the previous average slope as an updated average slope.

In this case, if the sample of the signal waveform intersects with the lower slope-tracing waveform, it has a special meaning.

Referring to FIG. 3 again, the sample 27 exhibits the crossover point with the lower slope-tracing waveform. Now, the crossover point where the lower slope-tracing waveform intersects with the signal waveform is defined as a slope-transition point, which implies a significant change in the average slope. Since the amplitude 27 of the signal waveform is higher than the height 28 of the lower slope-tracing waveform after the intersection, the procedure depicted in FIG. 2 is now applied.

In other words, since the amplitude of the signal waveform at samples thereafter is larger than the height 28 of the lower slope-tracing waveform, the lower slope-tracing waveform is updated with the amplitude 34 of the signal waveform.

Referring to FIG. 3, it should be noted that the height 14 of a lower slope-tracing waveform is maintained with the amplitude 9 at the slope-inversion point for the next three samples. If the samples of the signal waveform happen to exceed the lower slope-tracing waveform while the lower slope-tracing waveform is maintained, the procedure explained in FIG. 2 is then applied wherein the slope-inversion point is neglected and the signal waveform is assumed to increase.

Referring the FIG. 3, the lower slope-tracing waveform is maintained for three samples after the detection of a slope-inversion point. However, the number of samples can be arbitrarily chosen with different effects correspondingly.

In other words, the two neighboring waveforms can be considered as a single waveform or two individual waveforms in accordance with the number of samples, which can result in the effect of a low-pass filter.

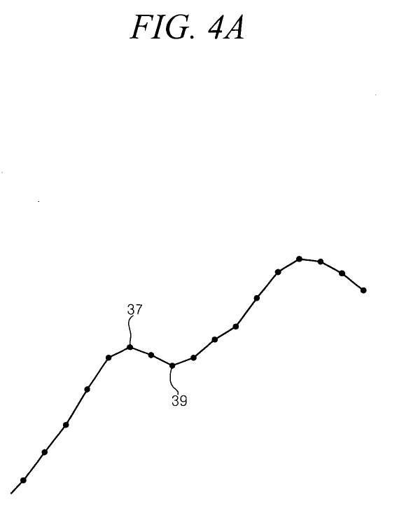

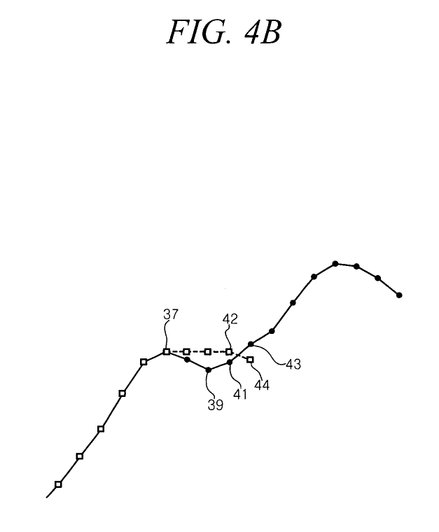

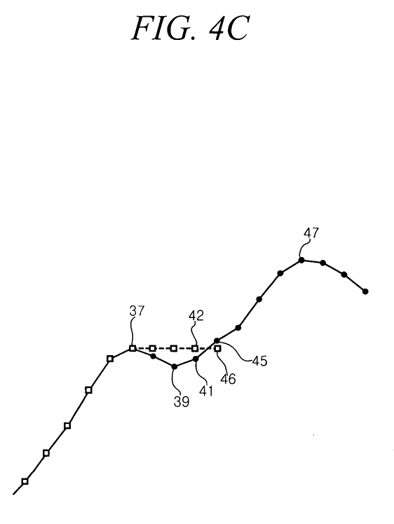

FIGS. 4A through 4C are schematic diagrams illustration the effect when the number of samples is varied wherein the lower slope-tracing waveform is maintained posterior to the detection of slope-inversion point in accordance with the present invention

Referring to FIG. 4A, it should be noted that the amplitude ceases to increase at the slope-inversion point 37 and the slope switches to a positive number at the second slope-inversion point 39.

Referring to FIG. 4B, the height 42 of the lower slope-tracing waveform is maintained for the three samples after the detection of a slope-inversion point 37.

In this case, the height 42 of the lower slope-tracing waveform is maintained up to the third sample 41, and is then updated with a new height 44 by subtracting with an average slope. Since the intersection occurs between the signal waveform and the lower slope-tracing waveform, a slope-transition point is determined and the interval between the first slope-inversion point 37 and a slope-transition point 43 is regarded as a single section.

Referring to FIG. 4C, it is noted the lower slope-tracing waveform is maintained for four samples (or even more than four) after the detection of the maximum 37.

As long as the samples of the signal waveform do not exceed the height 46 of the lower slope-tracing waveform after the detection of the first slope-inversion point 37 for four samples prior to the sample 45, samples prior to a sample 45, the procedure depicted in FIG. 2 is applied. In this case, the slope-inversion point 37 is neglected and the signal waveform is considered to increase continuously.

Through adjusting time (the number of samples) of maintaining the height of the lower slope-tracing waveform after the detection of a slope-inversion point, two successive waveforms can be either separated as two or regarded as one.

The method of partitioning a signal waveform by employing a lower slope-tracing waveform in accordance with the present invention performs the procedure disclosed to FIG. 2, FIG. 3, and FIG. 4, and the signal waveform is partitioned is consideration of a slope-inversion point and a slope-transition point.

The slope-transition point 9 depicted in FIG. 3 is a point where the lower slope-tracing waveform intersects the samples of the signal waveform from the negative to the positive and the signal waveform is maintained beneath the level of the lower slope-tracing waveform for three or K numbers of sample, and can be employed to determine the maximum of a signal waveform for certain interval.

The sample 27 of the signal waveform, as shown in FIG. 3, is a point where the signal waveform intersects with the lower slope-tracing waveform from the negative to the positive, and can be regarded as a slope-transition point where the signal waveform ceases to decreases for partitioning the signal waveform.

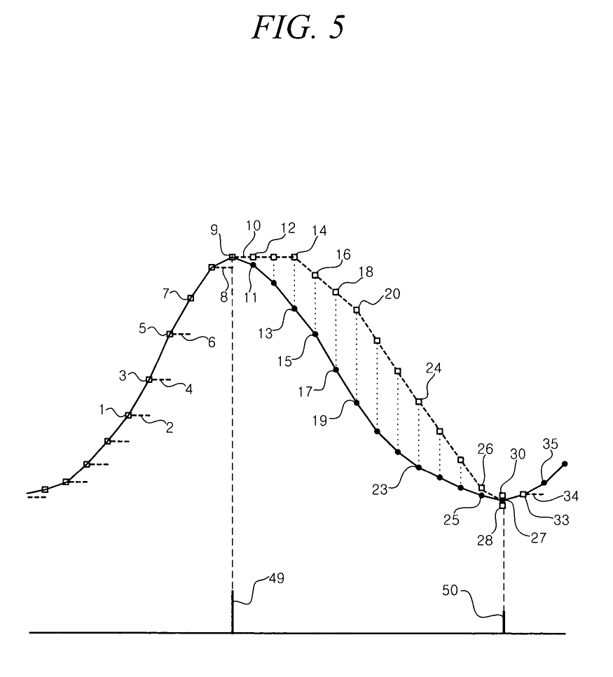

FIG. 5 is a schematic diagram illustrating a method of determining a slope-transition point by employing a lower slope-tracing waveform and preferred embodiments thereof. Referring to FIG. 5, it should be noted that the first bar 49 at the bottom means the slope-inversion point 9 of the signal waveform while the second bar 50 corresponds to the slope-change point. Further, it should be understood that the interval between those two bars should be regarded as a single interval. The amplitudes of those two bars 49, 50 are different form each other, which implies that the larger amplitude of the first bar 49 means a slope-inversion point while the smaller amplitude of the second bar 50 means a slope-transition point.

<Determination of a Slope-Inversion Point and a Slope-Change Point by an Upper Slope-Tracing Waveform>

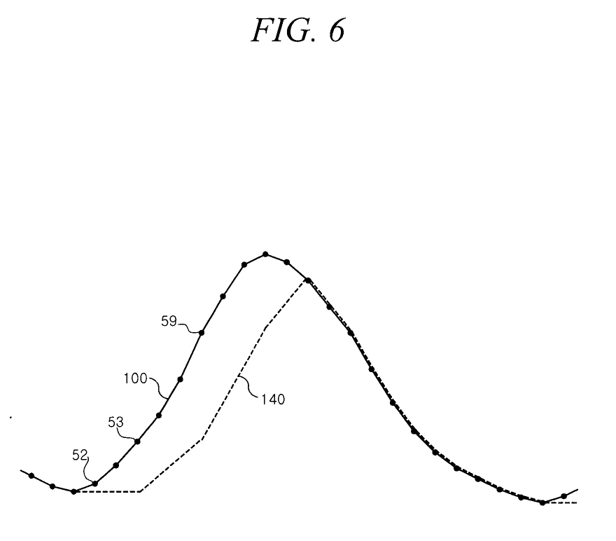

In the following a detailed description about an upper slope-tracing waveform will be given with reference to FIGS. 6 through 10 as another preferred embodiment in accordance with the present invention.

The behavior of the upper slope-tracing waveform is quite similar to that of the aforementioned lower slope-tracing waveform, while the difference between the two is that the upper slope-tracing waveform approaches the signal waveform downward from the top.

FIG. 6 is a schematic diagram illustrating a waveform-partitioning method with an upper slope-tracing waveform in accordance with the present invention. In FIG. 6 is shown a case when an upper slope-tracing waveform 140 is applied to a signal waveform 100. Referring to FIG. 6, a solid line 10 represents a signal waveform that needs to be partitioned, while the dots • 52, 53, 59 represents a sampled value (or amplitude at an instant under consideration) of the signal waveform and a dotted line 140 exhibits the behavior of an upper slope-tracing waveform.

Even if the upper slope-tracing waveform 140 depicted by dotted line 140 looks like approaching the signal waveform from the beneath, it is called “upper” slope-tracing waveform.

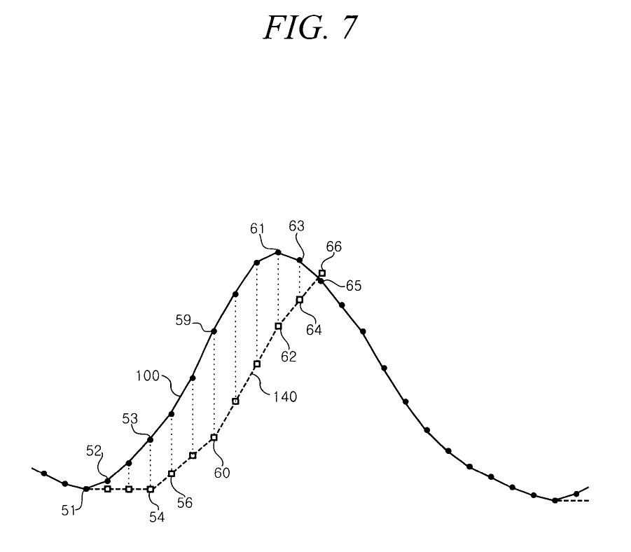

The behavior of the upper slope-tracing waveform, as shown in FIG. 7, can be classified as two cases depending upon the relative magnitude of the amplitude between the signal waveform an the slope-tracing waveform.

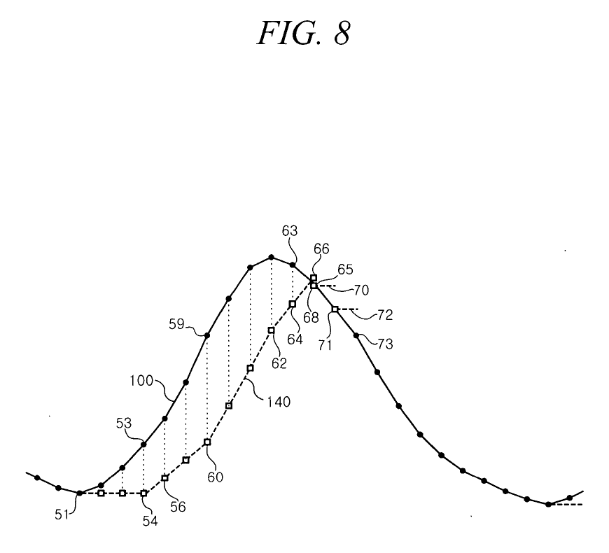

FIG. 7 illustrates a case when the amplitude of the signal waveform is greater than that of the upper slope-tracing waveform, while FIG. 8 corresponds to a case when the amplitude of the signal waveform is less than that of the upper slope-tracing waveform.

FIG. 7 is a schematic diagram illustrating the behavior of an upper slope-tracing waveform during the ascending stage where a signal waveform increases in accordance with the present invention.

Namely, FIG. 7 exhibits a case when the amplitude of a signal waveform is greater than that of an upper slope-tracing waveform.

Referring to FIG. 7, the waveform represented by a solid line 100 is a signal waveform which needs to be partitioned, while the sampled dots • 52, 53, 59, 16 represent the functional values of samples or the amplitude at an instant under consideration prior to the application of the upper slope-tracing waveform.

Moreover, the rectangles □ 54, 56, 60 denote the position of the upper slope-tracing after the samples are produced, while the dotted lines 140 denote the height of the upper slope-tracing waveform prior to sampling.

A detailed description of an upper slope-tracing waveform begins with a slope-inversion point 51 where the slope switches from the negative to the positive.

The upper slope-tracing waveform 140, which is updated with the slope-inversion point 51, maintains its height 54 up to the third sample 53.

If the amplitude of the signal waveform happens to be lower than the height of the upper slope-tracing waveform on the way to the third sample 53, the upper slope-tracing waveform is updated by a sample whose amplitude is lower than that of the upper slope-tracing waveform and the previously defined slope-inversion point is discarded.

However, if the signal waveform does not cross the upper slope-tracing waveform to go down below up until the third sample 53 from the slope-inversion point 51, the slope-inversion point is confirmed.

Further, the slope difference (or the amplitude difference) between the slope-inversion point 51 and the third sample 53 is calculated and divided by three in order to get an average slope per sample.

Now, the height of the upper slope-tracing waveform is updated with a new value 56 by adding the average slope per sample to the height of the upper slope-tracing waveform.

Now, the process of adding the average slope per sample to the upper slope-tracing waveform is kept on for the next samples after the average slope per sample is determined.

In the meanwhile, the amplitude of the signal waveform does not go below the height of the upper slope-tracing waveform for the next three samples, a new average slope per sample is updated and the upper slope-tracing waveform is updated by adding the average slope-per sample to the old upper slope-tracing waveform, which continues until the amplitude of a signal waveform becomes lower than the height of the upper slope-tracing waveform.

In FIG. 7 is shown a case where the height 54 of the upper slope-tracing waveform is maintained from the slope-inversion point 51 to the third sample 53 and the height 60 of the upper slope-tracing waveform is updated by adding the average slope per sample to the upper slope-tracing waveform.

In this case, the average slope per sample is updated again and added to the upper slope-tracing waveform on the way up to the next three samples 61.

In the meanwhile, the amplitude 65 of the signal waveform happens to be lower than that 66 of the upper slope-tracing waveform, a slope-transition point is determined and the upper slope-tracing waveform is updated with the amplitude the transition point.

As a preferred embodiment in accordance with the present invention, a new average slope per sample, which is calculated for every third sample, can be compared with the 50% value of the previously utilized average slope per sample.

If a new average slope per sample is lower than the previous one by more than 50%, the average slope per sample should be updated with a new number, which is 50% of the previous average slope per sample.

FIG. 8 is a schematic diagram illustrating a behavior of the upper slope-tracing waveform during the descending stage posterior to the slope-inversion pint in accordance with the present invention.

Referring to FIG. 8, the second part of the signal waveform demonstrates the behavior of the upper slope-tracing waveform when the amplitude of the signal waveform is lower than that of the slope-tracing waveform.

The first part of the waveform shown in FIG. 8 corresponds to the behavior illustrated in FIG. 7 while the second part illustrates the case when the amplitude of the signal waveform becomes lower than that of upper slope-tracing waveform.

Referring to FIG. 8, the solid line 100 denotes the signal waveform to be partitioned whereas the dotted line 140 denotes the upper slope-tracing waveform and the dots * 53, 59 imply the sampled value of the signal waveform, the rectangles □ 56, 60 denoting the height of each sample of the upper slope-tracing waveform.

The upper slope-tracing waveform is updated either with the previous sample or with the current sample depending upon the comparison in the amplitude.

Since the slope-transition point 65 lies below the upper slope-tracing waveform, the upper slope-tracing waveform is updated with a sample 68 and thereafter the height 70 is compared with the amplitude of the next sample 71.

In this case, since the height 70 of the upper slope-tracing waveform is larger than that of a sample 71, the upper slope-tracing waveform is updated with a signal sample 71 and maintains the height 72 in order to compared with next sample 73.

This process continues as long as the amplitude of a signal waveform lines above the upper slope-tracing waveform as shown in FIG. 7.

In the meanwhile, if the height of samples of a signal waveform lies below the upper slope-tracing waveform, on the contrary to the case shown in FIG. 7 wherein the height 54 of the upper slope-tracing waveform is maintained for the next three samples posterior to the slope-inversion point 51, the slope-inversion point is then discarded and the process illustrated in FIG. 8 is performed.

However, if the amplitude of a signal waveform happens to be lower than the height of the upper slope-tracing waveform during the ascending stage where the upper slope-tracing waveform increases with the average slope per sample after the period of maintaining the height for the three samples, the slope-inversion point is confirmed.

Although the number of samples where the amplitude of the upper slope-tracing waveform is maintained is three, one can choose the number as another preferred embodiment with a little bit different effect.

Depending upon the number of samples for maintaining the height of the slope-tracing waveform, two neighboring waveform can be considered either as one or two separate one, and thereby the effect of a low-pass filter can be expected.

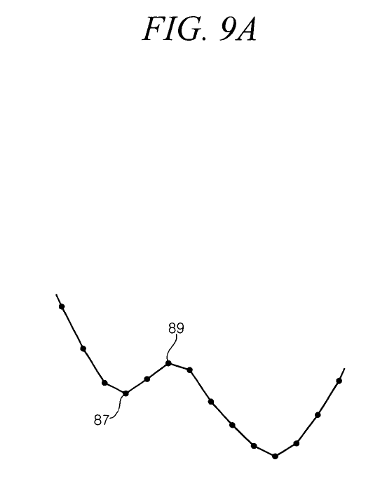

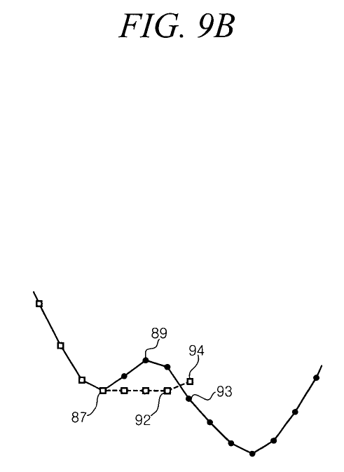

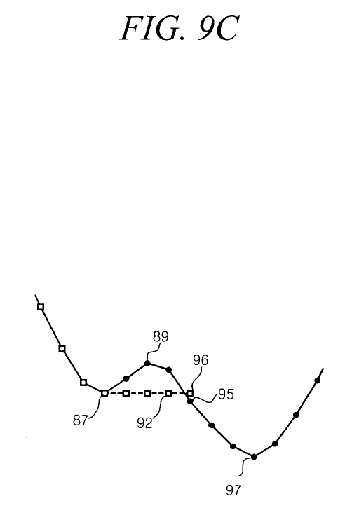

FIGS. 9A through 9C are schematic diagrams illustrating the dependence of the number of samples for maintaining the height of the slope-tracing waveform after the detection of the slope-inversion point.

Referring to FIG. 9A, a signal waveform increases' from the first slope-inversion point 87 up until the second slope-inversion point 89 after which the waveform decreases.

Referring to FIG. 9B, the height of the upper slope-tracing waveform is maintained with the amplitude 92 of the third sample after the first slope-inversion point 87 is reached. In this case, the height 94 of the upper slope-tracing waveform is updated by adding the average slope per sample, which is the average value of the three samples, to the height of the upper slope-tracing waveform.

Now, the next sample 93 is compared with the height 94 of the upper slope-tracing waveform. Since the signal waveform crosses down the upper slope-tracing waveform and the amplitude 93 lies below the height 94 of the slope-tracing waveform, the sample 93 is detected as a slope-transition point and separated from the subsequent waveform.

Referring to FIG. 9C, the height of the upper slope-tracing waveform is maintained up to the fourth sample 95 after the first slope-inversion point. In this case, the slope-inversion point 87 is discarded and the waveform is considered as decreasing because the amplitude 95 of the signal waveform becomes lower than that 96 of the slope-tracing waveform while the height of the slope-tracing waveform is kept constant.

Consequently, the up and downs of a signal waveform can be either separated or united depending upon how many samples are chosen form maintaining the height of the upper slope-tracing waveform with the amplitude of the slope-inversion point.

The number N of samples for maintaining the height of the upper slope-tracing waveform can be chosen under the consideration of the characteristic and/or the noise performance of the waveform, and further determined automatically.

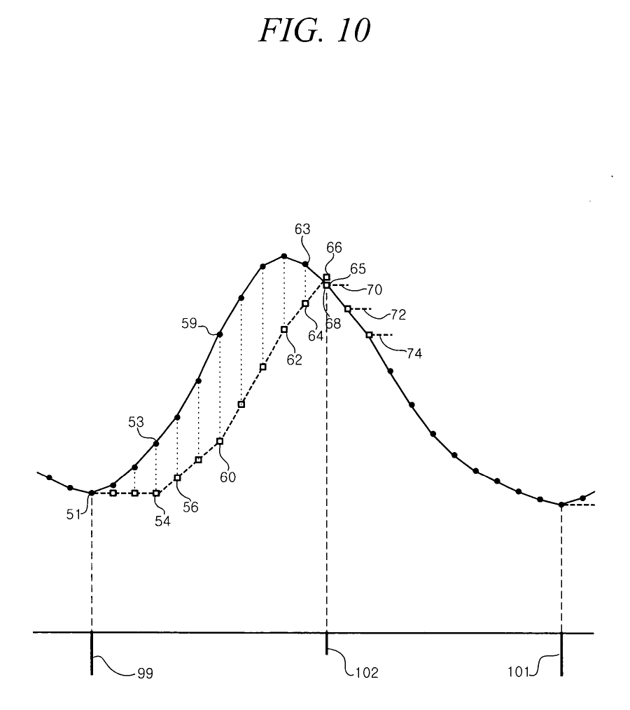

FIG. 10 is a schematic diagram illustrating a preferred embodiment for determining a slope-transition point and partitioning the waveform.

The first bar 99 shown in FIG. 10 implies the first slope-inversion point 51, while the second bar 102 with low height depicts a slope-transition point. The interval between those bars is considered as a single section.

In addition, the third bar 101 implies the second slope-inversion point of the signal waveform.

The method of partitioning a signal waveform with an upper slope-tracing waveform disclosed in the present invention performs the procedure illustrated in FIGS. 7, 8, and 9, and utilizes the slope-inversion point and the slope-transition point for partitioning the waveform.

The slope-inversion point 51, as shown in FIG. 7, is a point where the upper slope-tracing waveform starts to cross down the signal waveform and the height of the upper slope-tracing waveform of the upper slope-tracing waveform maintains its height for the next three or K samples, which is used for determining the minimum of a waveform for a particular section.

The sample 65 of the signal waveform depicted in FIG. 8 is a point where the signal waveform starts to go below the height of the upper slope-tracing waveform, which is considered as an ending point of increase and therefore a slope-transition point for the application of partitioning a waveform.

<Section-Partitioning Method of a Waveform>

The waveform partitioning method disclosed in the present invention is that a slope-inversion point is determined wherein the slope of a signal waveform changes its value from the positive and the negative and the amplitudes of the next three or N numbers of signal samples are lower than that of a point where the slope changes its value from the negative to the positive, while slope-transition point is determined by finding a point wherein a lower slope-tracing waveform keeps decreasing with an average slope per sample and finally becomes smaller than a sample of a signal waveform, and thereby those points are used for partitioning points as a reference.

The maximum sample 9 shown in FIG. 3 is a sample where a lower slope-tracing waveform has been smaller than the amplitude of a signal waveform and now starts to exceed, which determines a slope-inversion point where in the slope of a signal waveform changes from the positive to the negative. The sample 27 depicted in FIG. 3 is a point where the amplitude of a signal waveform has been smaller than the height of a lower slope-tracing waveform and then starts to exceed, which determines a slope-transition point by considering it as an ending point of decrease.

FIG. 5 demonstrates an example for the determination of slope-transition point by employing a lower slope-tracing waveform. The first bar 49, shown in FIG. 5, represents a slope-inversion point which is determined under the condition that the lower slope-tracing waveform maintains its height with the maximum 9 during three sampling instants, while the second bar 50 represents an instant when the height 28, which has been descending with an average slope per sample, becomes to be lower than a sample 27 and is regarded as a point where the slope changes very abruptly.

Thereafter, the slope-inversion point 51 of FIG. 7, at which the slope of the upper slope-tracing waveform changes from the negative to the positive and of which the slope is lower than those of the next three or N samples with the slope-transition point at which the upper slope-tracing waveform increases with an average slope and becomes larger than the amplitude of the signal waveform is a point where the amplitude of the upper slope-tracing waveform becomes lower than that of the signal waveform. Since the amplitude of the upper slope-tracing waveform is maintained during the next three samples, the slope-inversion point is now fixed. Further, the slope-transition point is fixed because the upper slope-tracing waveform increases with an average slope and then the height of the upper slope-tracing waveform becomes higher than that of the signal waveform. Thereby, the signal waveform is separated from the next signal interval.

FIG. 10 is a schematic diagram illustrating an embodiment of determining a slope-transition point by employing an upper slope-tracing waveform.

The first bar 99 of FIG. 10 denotes a slope-inversion point 51, which has been determined according to the condition that the amplitude of the upper slope-tracing waveform maintains its amplitude during the next three samples and thereby divides the signal waveform. The second bar 100 implies a slope-transition point where the amplitude 66 of the upper slope-tracing waveform becomes lower again than the samples 65, and thereby divides the signal waveform.

FIGS. 5 and 10 exhibits how to divide the signal waveform by employing the slope-inversion point and the slope-transition point. The bars shown in each figure denotes the partitioned point for the signal waveform.

The bars 49, 50 pointing to the positive direction denote the partitioned points, which are determined by a lower slope-tracing waveform, while the bars 99, 100, 101 pointing to the negative direction denote the partitioned points which are determined by an upper slope-tracing waveform.

The tall bar 49 of FIG. 5 denotes a slope-inversion point where the slope detected by the lower slope-tracing waveform changes from the positive to the negative, while the other bar 50 denotes a slope-transition point, which is detected by a lower slope-tracing waveform.

The tall bars 99, 101 denote the slope-10 inversion points where the slope, detected by an upper slope-tracing waveform, changes from the negative to the positive, while the other bar 100 denotes a slope-transition point detected by an upper slope-tracing waveform.

The waveform partitioning method as set forth in the foregoing upper and lower slope-tracing waveforms has been applied in such a way that the time axis of the slope-tracing waveform increases.

This can be utilized either for the real-time recognition of the signal waveform or for the stored waveform.

In case when the recognition of a certain waveform from the stored signal waveform is needed, the upper and lower slope-tracing waveforms can be applied in the reverse time axis. In other words, the stored waveform can be partitioned in accordance with the present invention by applying the slope-tracing waveforms from the final toward the initial in the reverse time axis. In order to divide to signal waveform more accurately, the aforementioned slope-tracing waveform can be applied both directions of the time axis. In other words, both the upper slope-traction waveform and the lower slope-tracing waveform are utilized in a forward time axis and thereafter in a reverse time axis.

More preferably, the direction in time axis for applying the slope-tracing waveform can be alternated, if need. Namely, for instance, one can apply the upper and lower slope-tracing waveforms in the positive direction of time axis for certain period of samples. Now, when either a slope-inversion point or a slope-transition point is reached, the direction of time axis for applying the slope-tracing waveforms can be switched until either a new slope-transition point or a slope-inversion point is detected. In this case, if the time for applying the slope-tracing waveforms in the reverse direction is shorter than the sampling period, it can be applied in real time.

In the above explanation, the waveform partitioning method by upper and lower slope-tracing waveforms defines the spacing between the slope-inversion point and the neighboring slope-transition point as a single interval. More preferably, however, the interval between the left and right slope-transition points with respect to a slope-inversion point as a center can be regarded as a single point. Although this can be applied to the slope-inversion point and the slope-transition point determined either by an upper slope-tracing waveform or by a lower slope-tracing waveform in a separate manner, in can be also applied to a slope-inversion point and a slope-transition point mixed from the two slope-tracing waveforms.

The interval partitioned by the lower slope-tracing waveform and the upper slope-tracing waveform, as shown in FIGS. 5 and 10, can be amended as the following, if needed.

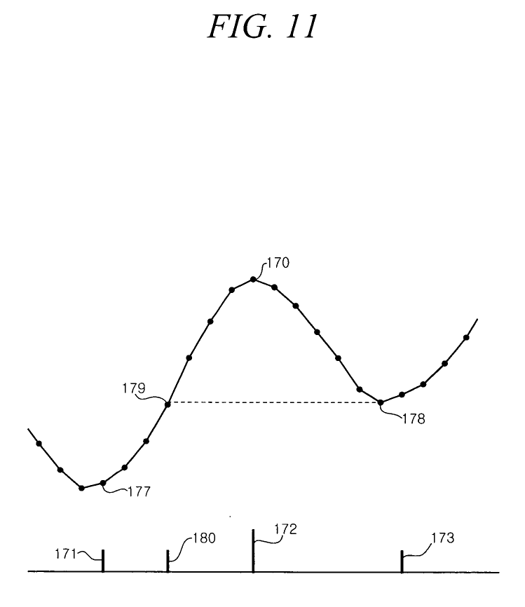

FIG. 11 is a schematic diagram illustrating a partitioned waveform determined by a lower slope-tracing waveform. The first bar 171 and the last bar 173 depicted in FIG. 11 correspond to a slope-transition point determined by a lower slope-tracing waveform, while the third bar 172 corresponds to a slope-inversion point determined by a lower slope-tracing waveform.

It is noted that there is a significant difference in the amplitude 177, 178 of the signal waveform between at the left slope-transition point 171 and at the right slope-transition point 173 of the slope-inversion point 172 that is determined by the slope-tracing waveform.

In this case, if the difference in the amplitude between at the right slope-transition point 173 and at the left slope-transition point 171 exists by more than Y percent, the slope-transition point 171, 173, which have been detected by a lower slope-tracing waveform, are still used.

In the opposite case, as shown in FIG. 11, the position of the slope-transition point is adjusted and the partitioned interval is modified.

As a preferred embodiment in accordance with the invention, a slope-transition point 173 whose amplitude 178 is close to that 170 of the signal waveform at the slope-inversion point 172 is selected. Moreover, a slope-transition point 180 for adjusting a sampling instant can be determined by finding a sample 179 whose amplitude is most close to the that 178 of the slope-inversion point 173 in order to amend the interval partitioned by the lower slope-tracing waveform.

Preferably, Y can be chosen in the numbers between 30 and 90 according to the characteristics of the signal waveform. Especially for the physiological signal of a living body, 70% can be chosen for Y.

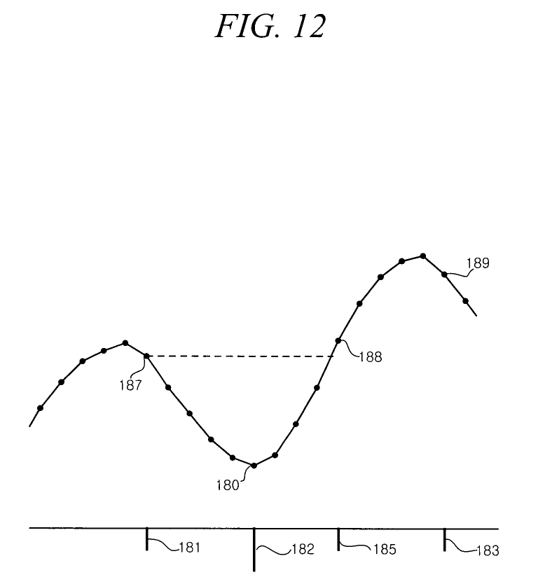

FIG. 12 is a schematic diagram illustrating a waveform partitioned with points determined by an upper slope-tracing waveform.

The first bar 181 and the last bar 183 of FIG. 12 represent slope-transition points from the upper slope-tracing waveform, while the second bar 182 is a slope-inversion point. There is a significant difference in the amplitude 187, 189 between the left slope-transition point 181 and the right slope-transition point 183 with respect to the slope-inversion point 182 from the upper slope-tracing waveform.

In this case, the difference between the amplitude 180 of the signal waveform at the slope-inversion point 182 and the amplitudes 187, 189 at the slope-transition points is calculated, respectively.

Furthermore, if the ratio between the larger amplitude and the smaller amplitude is more than Y %, the slope-transition points 181, 183 determined by the upper slope-tracing waveform should continue to be utilized.

However, if the opposite is true, the position of the slope-transition points should be adjusted as shown is FIG. 12 in order to amend the partitioned interval.

The interval determined from the lower slope-tracing waveform can be amended by selecting a slope-transition point 181 having an amplitude 187 that is close to the amplitude 180 of the signal waveform at the slope-inversion point, and defining a sampling instant as a slope-transition point 185 wherein the amplitude 188 of the opposite signal waveform is close to the amplitude 187 of a chosen slope-transition point 181.

In the meanwhile, any number between 30 and 90 can be chosen for Y. Preferably, 70 can be used as Y for the physiology signal. The amendment explained in the foregoing can be selectively applied, if needed.

<Characterization of Partitioned Waveform>

In the followings, a detailed description will be made for characterizing the partitioning points of the signal waveform which has been determined from the slope-inversion point and the slope-transition point with upper and lower slope-tracing waveforms.

As a first embodiment in accordance with the present invention, and interval can be characterized by indication the area at the end of the interval, which is obtained from an integration of the waveform between the partitioning points.

The area of the signal waveform at each interval is obtained by subtraction the sampled values in the interval from the amplitude of the signal waveform at a slope-transition point, followed by summing the subtracted values.

In addition, if the interval between the right and the left slope-transition points with a center at the slope-inversion point is defined as a single point, either the sum or the of the pair first part and the second part calculated from the above can be utilized for the characterization of the waveform.

As a second embodiment in accordance with the invention, the amplitudes in the interval that is partitioned from the partitioning points can be utilized for the characterization. Here, the amplitude is defined as the subtraction of the amplitude at a slope-transition point from the amplitude at a slope-inversion point.

In addition, in case when the interval of the signal waveform is defined as an interval between the left slope-transition point and the right slope-transition point with a center at a slope-inversion point, the sum of the amplitudes of the first part and the second part can be utilized as well as the pair of the amplitudes.

As a third preferred embodiment in accordance with the present invention, the time interval partitioned by the slope-partitioning points is calculated and is characterized. The time interval is defined as a time difference between the beginnings to the end of the interval.

Additionally, when a signal waveform interval is defined as spacing between the left and the right slope-transition point with a center at an slope-inversion point, either the sum or the pair themselves of the first part and the second part can be utilized for the characterization of the interval.

The above-mentioned three embodiments can be applied either separately or simultaneously. In other words, either the area or the amplitude calculated in accordance with the present invention can further reduce the characteristics of the signal waveform by partitioning or multiplying in time interval.

Although the invention has been illustrated and described with respect to exemplary embodiments thereof, it should be understood by those skilled in the art that various other changes, omissions and additions may be made therein and thereto, without departing from the spirit and scope of the present invention.

Therefore, the present invention should not be understood as limited to the specific embodiment set forth above but to include all possible embodiments which can be embodies within a scope encompassed and equivalents thereof with respect to the feature set forth in the appended claims.

### INDUSTRIAL APPLICABILITY

As explained in the foregoing, the present invention can be useful for partitioning the signal waveform in such a way that the partitioned waveform is suitable to the recognition of a signal with the upper and lower slope-tracing waveform.

More particularly, the waveform partitioning method in accordance with the present invention can be employed for the physiology signal of the medical instrument.

What is claimed is:

1. A method of partitioning a sampled signal waveform into several sections each of which includes a multiple of samples with a tracing waveform, comprising steps of:
(a) updating the functional value of said tracing waveform at an (n+1)-th sample with the amplitude of said signal waveform at an (n+1)-th sample if the functional value of said tracing waveform at an n-th sample is smaller than the amplitude of said signal waveform at an (n+1)-th sample;
(b) comparing the functional value of said tracing waveform at an n-th sample with that at an (n−1)-th sample of said tracing waveform if the functional value of said tracing waveform at an n-th sample is greater than or equal to the amplitude of said signal waveform at an (n+1)-th sample;
(c) either maintaining the functional value of said tracing waveform at an (n+1)-th sample with that at an n-th sample of said tracing waveform in case when the functional value of said tracing waveform at consecutively foregoing samples including an n-th, an (n−1)-th, an (n−2)-th, . . . , has been kept constant wherein the number of samples is less than a predefined number k, or updating the functional value of said tracing waveform at an (n+1)-th sample by subtracting the functional value of said tracing waveform at the n-th sample with an average slope between the n-th sample and the (n−k)-th sample that is regarded as a slope-inversion point in the case when the number of samples is more than or equal to said predefined number k at the step of (b);
(d) updating the functional value of said tracing waveform at an (n+1)-th sample by subtracting a first slope from the functional value of said tracing waveform at an n-th sample if the value of said tracing waveform at an n-th sample is different from that at an (n−1)-th sample and the number of samples including the n-th, (n−1)-th, an (n−2)-th, . . . of which the value has been decreasing with the same slope (said “first slope”) is less than a predefined number L, or by subtracting a second slope from the functional value of said tracing waveform at an n-th sample if the number of samples decreasing with said first slope is greater than or equal to said predefined number L and the average slope (“a second slope”) between the n-th sample and the (n−L)-th sample is steeper than said first slope multiplied by a predefined rate (X %), or by subtracting a first slope multiplied by said predefined rate (X %) from the functional value of said tracing waveform at an n-th sample if said second slope is less steep than said first slope multiplied by said predefined rate (X %) at step of (b); and
(e) regarding the (n+1)-th sample as a slope-transition point and regarding the interval between said slop-changing point and said slope-inversion point as a single section if the functional value of said tracing waveform at an (n+1)-th sample is lees than or equal to the value of said signal waveform at an (n+1)-th sample and thereby the two waveforms intersect.

2. A method of partitioning a sampled signal waveform into several sections each of which includes a multiple of samples with a tracing waveform, comprising steps of:
(a) updating the functional value at an (n+1)-th sample of said tracing waveform with the amplitude of said signal waveform an (n+1)-th sample if the functional value of said tracing waveform at an n-th sample is larger than the amplitude of said signal waveform at an (n+1)-th sample;
(b) comparing the functional value of said tracing waveform at an n-th sample with that at an (n−1)-th sample of said tracing waveform if the functional value of said tracing waveform at an n-th sample is smaller than or equal to the amplitude of said signal waveform at an (n+1)-th sample;
(c) either maintaining the functional value of said tracing waveform at an (n+1)-th sample with that at an n-th sample in case when the functional value of said tracing waveform at consecutively foregoing samples including an n-th, an (n−1)-th, an (n−2)-th, . . . , has been kept constant wherein the number of times is less than a predefined number k, or updating the functional value of said tracing waveform at an (n+1)-th sample with an (n+1)-th sample by adding the functional value of said tracing waveform at the n-th sample with an average slope between the n-th sample and the (n−k)-th sample which is regarded as a slope-inversion point in case when the number of samples is more than or equal to said predefined number at the step of (b);
(d) updating the functional value of said tracing waveform at an (n+1)-th sample by adding a first slope from the functional value of said tracing waveform at an n-th sample if the value of said tracing waveform at an n-th sample is different from that at an (n−1)-th sample and the number of samples including the n-th, (n−1)-th, an (n−2)-th, of which the value has been increasing with the same slop (said “first slope”) is less than a predefined number L, or by adding a second slope from the functional value of said tracing waveform at an n-th sample if the number of samples increasing with said first slope is greater than or equal to said predefined number L and the average slope (“a second slope”) between the n-th sample and the (n−L)-th sample is steeper than said first slope multiplied by a predefined rate (X %), or by adding a first slope multiplied by said predefined rate (X %) from the functional value of said tracing waveform at an n-th sample if said second slope is less steep than said first slope multiplied by said predefined rate (X %) at step of (b); and
(e) regarding the (n+1)-th sample as a slope-transition point and regarding the interval between said slop-changing point and said slope-inversion point as a single section if the functional value of said tracing waveform at an (n+1)-th sample is greater than or equal to the value of said signal waveform at an (n+1)-th sample and thereby the two waveforms intersect.

3. The method as set forth in claim 1 or claim 2 further comprising steps of: subtracting the amplitude of said signal waveform at slope-transition point from the amplitude of said signal waveform at each sample in said partitioned section and summing the values of subtraction at each sample for the calculation of the area of said partitioned section.

4. The method as set forth in claim 1 or claim 2 wherein the interval between the left slope-transition point and the right slope-transition point with respect to said slope-inversion point as a center is defined as a single section, further comprising steps of:
calculating the area of the left and right part of the waveform in said partitioned section; and
characterizing said partitioned section by summing the calculated area of the left and right part of the waveform.

5. The method as set forth in claim 1 or claim 2 wherein the interval between the left slope-transition point and the right slope-transition point with respect to said slope-inversion point as a center is defined as a single section, further comprising steps of:
calculating the area of the left and right part of the waveform in said partitioned section; and
characterizing said partitioned section by coupling a pair of the calculated area of the left and right part of the waveform.

6. The method as set forth in claim 1 or claim 2 further comprising a step of characterizing the waveform of the partitioned section by calculating the difference in amplitude of the signal waveform at each samples in the partitioned section.

7. The method as set forth in claim 1 or claim 2 further comprising a step of calculating the difference in amplitude in said partitioned section by subtracting the amplitude of a slope-transition point from that of a slope-inversion point.

8. The method as set forth in claim 1 or claim 2 wherein the interval between the left slope-transition point and the right slope-transition point with respect to a slope-inversion point as a center is regarded as a single section, further comprising a step of characterizing said section by subtracting the amplitude of the slope-inversion point from that of each slope-transition point and summing the subtracted value.

9. The method as set forth in claim 1 or claim 2 wherein the interval between the left slope-transition point and the right slope-transition point with respect to a slope-inversion point as a center is regarded as a single section, further comprising a step of characterizing said section by subtracting the amplitude of the slope-inversion point from that of each slope-transition point and coupling a pair of the subtracted value.

10. The method as set forth in claim 1 or claim 2 further comprising a step of defining a time interval by calculating a time difference between the initial point and the final point of said partitioned section determined by said slop-changing point and said slope-inversion point.

11. The method as set forth in claim 1 or claim 2 wherein a section is defined as an interval between the left slope-transition point and the right slope-transition point with respect to a slope-inversion point as a center, further comprising a step of characterizing the time interval of said section by calculating the time difference between the initial and final points and summing the values.

12. The method as set forth in claim 1 or claim 2 wherein a section is defined as an interval between the left slope-transition point and the right slope-transition point with respect to a slope-inversion point as a center, further comprising a step of characterizing the time interval of said section by calculating the time difference between the initial and final points and coupling a pair of said values.

13. The method as set forth in claim 1 or claim 2 wherein the amplitude of the slope-inversion point and of the left and right slope-transition points is detected, further comprising steps of:
selecting a slope-transition point among the two whose amplitude is more similar to that of said slope-inversion points; and
amending said partitioned section by selecting a sample of said signal waveform as another slope-transition point whose amplitude is the most similar to that of the selected slop-changing point and whose position is the most close to the mirror location of said selected slope-transition point with respect to said slope-inversion point if the difference in amplitude between the left and right slope-transition points exist by less than a predetermined amount (Y %).
